(19) United States
(12) Patent Application Publication
(10) Pub. No.: US 2024/0216642 A1
(43) Pub. Date: Jul. 4, 2024

(54) SYSTEM AND METHOD FOR BIOFEEDBACK

(71) Applicant: ALPHABEATS WORKS B.V., Eindhoven (NL)

(72) Inventors: Johannes Bernardus KORTAS, Eindhoven (NL); Jurrien Hein Bernard Franco VELLEMA, Eindhoven (NL); Han DIRKX, Eindhoven (NL)

(21) Appl. No.: 18/288,966

(22) PCT Filed: May 2, 2022
(86) PCT No.: PCT/NL2022/050238

§ 371 Date: Oct. 30, 2023

(30) Foreign Application Priority Data
Apr. 30, 2021 (NL) 2028120

(51) Int. Cl. A61M 21/00

(52) U.S. Cl. CPC A61M 21/00; A61M 2021/0027; A61M 2021/005; A61M 2205/70

(57) ABSTRACT

System for biofeedback, comprising: at least one input apparatus configured for capturing at least one bio-signal of a user; a processing module configured for adjusting a sensory signal relative to a default setting of the sensory signal; a signal interface configured for outputting the adjusted sensory signal to a signal playback device arranged for being perceivable to the user; wherein the adjusting comprises: determining at least one characteristic based on the at least one bio-signal, over a calibration period of at least 10 seconds; adjusting the sensory signal based on the at least one characteristic, using a machine learning procedure or based on an output from a pre-trained machine learning system.

### FIELD OF THE INVENTION

The present disclosure is generally related to biofeedback. In particular, it relates to a system for biofeedback, a method for biofeedback, a method for training a recommender system, a recommender system, a method for providing an equalizer, use of such an equalizer, and a computer-readable data carrier.

### BACKGROUND

Conventional approaches to biofeedback exist, wherein a bio-signal of a user is measured and wherein the user actively performs relaxation or meditation techniques, in particular in order to relieve stress.

Another known approach exists, wherein a bio-signal of a user is measured, and wherein a filter is applied to an audio signal for variably filtering the audio signal by modifying a cut-off frequency in response to the bio-signal, in order to reduce human stress.

### SUMMARY

It is a drawback of the above-described approaches that users find it very difficult to keep using them in the required instructive way over a long time. This is an important drawback, since it is well-known that human stress can be much more effectively reduced with any treatment if that treatment is used in a subconscious way and/or over a long time

It is a general aim of embodiments according to the present disclosure to provide biofeedback in order to enhance measurable mental state focus of a user, in particular for meditative improvement or performative improvement. In qualitative terms, mental state, or mental health state, may be related to increased feelings of concentration, control, relaxation and flow state of mind, and to decreased feelings of stress, or insomnia.

The skilled person will understand that focus can be measured in a variety of ways. e.g. by measuring and analysing certain brainwaves (i.e. neural oscillations, which is a form of brain activity) of the user. It is well-known that alpha waves correlate with non-arousal, creativity and relaxation, whereas beta waves correlate with arousal, anxiety, stress. and actively engaged in mental activities.

It is a more specific aim of embodiments according to the present disclosure to provide biofeedback in such a way as to stimulate users to keep using the biofeedback over a long time.

It is an insight of the inventors that users may be stimulated to keep using biofeedback over a long time by better tailoring the biofeedback to them. In this context, “tailoring” may mean adapting based on the user.

According to a first aspect according to the present disclosure, there is provided a system for biofeedback, comprising:
at least one input apparatus configured for capturing at least one bio-signal of a user;
a processing module configured for adjusting a sensory signal relative to a default setting of the sensory signal;
a signal interface configured for outputting the adjusted sensory signal to a signal playback device arranged for being perceivable to the user;
wherein the adjusting comprises;
determining at least one characteristic based on the at least one bio-signal;
adjusting the sensory signal based on the at least one characteristic, using a machine learning procedure or based on an output from a pre-trained machine learning system.

Moreover, the determining spans a calibration period of at least 10, preferably at least 20, seconds prior to the adjusting.

By basing the adjusting on the at least one characteristic, which in turn depends on the at least one bio-signal of the user, it is possible to better tailor the biofeedback to the specific user. Due to this better tailoring, the user may experience yearning for the biofeedback. In this manner, the user may be more likely to keep using the biofeedback over a long time. Moreover, by introducing a calibration period, it is possible to better individualize for the user, not only by allowing the at least one bio-signal to converge more accurately, but also by stabilizing any considered adjustment before effecting it. It is believed that the benefit of using this calibration period stems from the relatively slow rate of change of bio-signals of the user, in the sense that it takes some time for the human physiology to adapt and that the system thus is able to take this time better into account.

In the context of the present disclosure, the term calibration period may be taken to refer to a period of monitoring without actively adjusting.

The input apparatus may comprise a sensor. Additionally or alternatively, the input apparatus may be coupled with a separate sensor.

In this context, a “characteristic” may mean a distinguishing trait, quality, or property, applying to something that distinguishes or identifies a person or thing or class. In the case of a signal, a characteristic may be any signal characteristic available via signal processing. In the case of a person, a characteristic may be termed a user characteristic. Such a characteristic may be broad, e.g. a gender or an age group, or it may be specific, e.g. an age, a weight or a height. Moreover, a characteristic may also be time-specific, for example it may indicate whether a person is at some point in time relaxed or excited, or it may even help to discern that the person is distracted, happy or fearful.

In this context, “adjusting” may mean modifying the original signal (optionally including adding an extra signal), but may also mean maintaining the original signal and adding an additional signal.

In this context, “adjusting a sensory signal relative to a default setting of the sensory signal” may be taken to mean that an original version of the sensory signal is adjusted to become a new, different version of that sensory signal and/or may be taken to mean that a pre-set configuration of the original sensory signal is adjusted in order to produce an altered sensory signal as compared to the original sensory signal.

It is a further aim of at least some embodiments according to the present disclosure to balance any adjustment to the sensory signal such that on the one hand the user cannot notice or can only minimally notice the adjustment, but on the other hand the adjustment leads to measurable or preferably maximal impact on the user. In other words, it is a further aim of at least some embodiments according to the present disclosure to operate from the point of view of the user as subconsciously as possible.

The effect of embodiments according to the present disclosure may be measured by capturing bio-signals of the users. Example bio-signals for this purpose are described below. Additionally or alternatively to this way of measuring impact on focus, impact may also be measuring using a subjective opinion score of the user.

The at least one bio-signal captured by the at least one input apparatus preferably comprises at least one of the following examples. Example bio-signals may include brainwaves, as described above, which can be captured via e.g. EEG (electroencephalography) or ECoG (electrocorticography). Example bio-signals may additionally or alternatively include at least one of the following:
muscular activity, which may be determined via e.g. EMG (electromyography);
heart rate, which may be determined via e.g. ECG (electrocardiogramar a pulse wave signal derived from a PPG (photoplethysmograph);
HRV (heart rate variability), which may be determined via e.g. ECG, blood pressure, ballistocardiograms, or a pulse wave signal derived from a PPG (photoplethysmograph);
breath rhythm;
blood pressure;
body temperature;
gas content of blood;
perspiration, e.g. a flow rate, and/or cortisol concentration, etc.;
electrodermal activity (EDA), e.g. a skin conductance, GSR (galvanic skin response), etc.;
facial expression;
dexterity performance parameter, e.g. typing or handwriting or handdrawing behaviour; and
voice pattern.

Breath rhythm and heart rate (and successively HRV) are currently preferred. Creating a state of coherence, in which heart rate (and successively HRV) and breath rhythm are synchronized is known to create more alpha waves in the brain, corresponding with a state of relaxation. In this context, the skilled person will appreciate that more alpha waves and a high HRV may indicate that a user has enhanced well-being and balance of body and mind (dominated by parasympathetic nervous system), whereas beta waves and a low HRV may indicate that a user has a more rigid heart rate, which may indicate higher stress (dominated by the sympathetic nervous system).

Preferably non-invasive scanning techniques may be used; therefore, invasive techniques such as ECoG (electrocorticography) are not preferred, because the invasion of the user's body may likely reduce the user's positive feelings of well-being and balance of body and mind.

Currently, body temperature is less preferred because the timescale at which it fluctuates is considered too slow. However, in principle, it may be used.

The at least one bio-signal may be a real-time signal or a sample. In this context, a sample is a value at a discrete point in time. It is advantageous if the at least one bio-signal is a real-time signal because this directly shows the user's real-time physiology. Alternatively, samples may be used, because although samples have some latency compared to a real-time signal, this latency may be disregarded since the physiology of the user changes only within certain limits over a time scale on the order of several seconds, e.g. 2-30 seconds. Most preferably, a sample of at most 15-20 seconds old may be used without sacrificing insight into the user's physiology. In a further development, burst-mode samples may be used, wherein a plurality of samples collected over a time period are bundled and captured in a burst. In this manner, technical feasibility may be improved over bandwidth-limited channels.

Preferably, the at least one characteristic is a signal characteristic of the at least one bio-signal, and/or the at least one characteristic is a user characteristic selected for distinguishing a group to which the user belongs among a plurality of groups or distinguishing the user individually from other users.

In this manner, the adjusting may correspond to the user, while being more cost-effective because the adjusting is only done to group level. Alternatively, if the adjusting is to the level of the individual user, the adjusting may be perfectly tailored.

Example groups of users may comprise e.g. children, young adults, adults, pregnant women, elderly, attention-disordered people, etc. Particular groups among these groups may be physiologically limited, e.g. due to limited bandwidth HRV, e.g. pregnant women in function of progression of their pregnancy, or e.g. elderly people who can hear less well in higher frequencies.

Preferably, the adjusting is further based on at least one pre-set, wherein the at least one pre-set comprises at least one of: an equalizer; a genre setting of the equalizer such as rock, jazz, eighties, etc.; and a timbre. For visual sensory signals, the timbre may relate to a setting of brightness and/or a setting of colour. The pre-set may preferably be manually set by the user prior to the adjusting, or it may be a default pre-set set by an operator of the system.

In this manner, the adjusting can start from a readily available default. Moreover, in this manner, this default can be assumed to be pleasing to the user.

The processing module is preferably configured for:
defining the at least one characteristic determined at the end of the calibration period as a reliable level of the at least one characteristic;
during the adjusting, detecting whether a reliability of the at least one characteristic becomes less than the reliable level; and
upon detecting that the reliability of the at least one characteristic becomes less than the reliable level, temporarily suspending the adjusting of the sensory signal until another calibration period of at least 10, preferably at least 20, seconds has passed.

In this manner, if during a session the reliability of the at least one characteristic drops below the reliable level as defined at the end of the original calibration period, the system is able to detect this and is able to recalibrate, using another calibration period of similar duration, to ensure that the at least one characteristic again reaches an acceptable level of reliability.

The sensory signal may preferably be a media signal, as described below. Alternatively, a sensory signal may also be another type of sensory signal, including but not limited to:
light, e.g. defined as a frequency or frequency range of electromagnetic radiation, an amplitude of lumen value, as a single or composite colour value, or as a combination thereof;
temperature;
smell;
haptics, i.e. tactile stimulation; and
in limited measure even pain, e.g. via acupuncture or very low-power electrical stimulation.

A media signal may comprise at least one type of signal, i.e. a single type of signal or at least two types of signals. The latter option may be termed a multimedia signal. For example, a signal comprising only an audio signal (i.e. audible components) is a media signal, a signal comprising only a video signal (i.e. visible components coming from an electrical signal designed to produce an image or a sequence of images) is a media signal, and a signal comprising both an audio and a video signal is a media signal and more specifically a multimedia signal.

The term sensory may be taken to mean relating to the senses, in particular detectable by the senses. Preferably, the sensory signal is a media signal, and the sensory signal is based on, preferably selected from, a plurality of media signals belonging to a predetermined media library of the user.

Advantageously, this may be favourite audio (music) or favourite video (films). The library may e.g. be pre-determined initially, but may also be determined in ongoing operation as well, to ensure ongoing correspondence with the user's preferences. The library may preferably be determined by analysing media content stored on a media device of the user, e.g. a local device of the user, or a NAS (network attached storage) on a LAN (local area network) of the user; and/or by analysing media content associated with the user on an internet streaming service, e.g. Spotify®, Last.fm™, Pandora®, Apple Music®, etc.

Preferably, the sensory signal comprises an audio signal, and wherein the adjusting comprises at least one of:
amplifying or weakening audio amplitudes of the audio signal over at least 3, preferably at least 5, frequency bands; and
removing or adding at least one audio tone from or to at least one frequency band.

Preferably, the adjusting is performed in such a way that hearable audio distortion is prevented near edges of the frequency bands and that higher harmonic frequencies of the audio signal are maintained.

In this manner, the audio signal may be kept pleasing to the user, which may increase the probability that the user will keep using the biofeedback over a long time. Preferably, the sensory signal comprises a video signal, and the adjusting comprises at least one of:
amplifying or weakening at least one of the following: a light output value (e.g. brightness); a colour value; a video framerate; a video blur; and a video focus;
removing or adding at least one video frame of the sensory signal; and
removing or adding at least one video component from or to at least one video frame of the sensory signal.

The at least one video component may be any conceivable component of the video signal, including but not limited to regions of the video frame or objects visible in the video frame.

In a particularly preferred embodiment, the adjusting is narrowed down over time in terms of minimum and maximum bandwidth, via a progressive average.

In this manner, the adjusting may converge over time, in order to reduce abruptness.

In a further developed embodiment, the narrowing down may take into account the determined at least one characteristic and/or at least one predetermined property of the user, such as a user group to which the user belongs.

Preferably, the adjusting is based on different cut-off frequencies and/or different attenuation values for different individual users and/or for different groups of users.

Preferably, the adjusting is further based on contextual data, such as location, timestamp, activity levels of a past time period, season, weather, etc.

Preferably, the sensory signal comprises a light signal, and the adjusting comprises at least one of:
changing a combination of electromagnetic frequencies of the light signal;
amplifying or weakening at least one light amplitude over at least one frequency range of the light signal; and
varying at least one light amplitude of the light signal over a time pattern.

According to a second aspect according to the present disclosure, there is provided a method for biofeedback, comprising:
capturing at least one bio-signal of a user;
adjusting a sensory signal relative to a default setting of the sensory signal;
and
outputting the adjusted sensory signal to a signal playback device arranged for being perceivable to the user;
wherein the adjusting comprises:
   determining at least one characteristic based on the at least one bio-signal; and
   the sensory signal based on the at least one characteristic, using a machine learning procedure or based on an output from a pre-trained machine learning system. Moreover, the determining spans a calibration period of at least 10, preferably at least 20, seconds prior to the adjusting.

The skilled person will appreciate that analogous considerations and advantages may apply to the method as for the above-described system, mutatis mutandis.

Preferably, the method comprises:
defining the at least one characteristic determined at the end of the calibration period as a reliable level of the at least one characteristic;
during the adjusting, detecting whether a reliability of the at least one characteristic becomes less than the reliable level; and
upon detecting that the reliability of the at least one characteristic becomes less than the reliable level, temporarily suspending the adjusting of the sensory signal until another calibration period of at least 10, preferably at least 20, seconds has passed.

Preferably, the at least one bio-signal comprises at least one of the following:
brainwaves;
a muscular activity;
a heart rate;
a heart rate variability;
a breath rhythm;
a blood pressure;
a body temperature;
a gas content of blood;
a perspiration;
an electrodermal activity
a facial expression;
a dexterity performance parameter; and
a voice pattern.

Preferably, the at least one characteristic is a signal characteristic of the at least one bio-signal and/or the at least one characteristic is a user characteristic selected for distinguishing a group to which the user belongs among a plurality of groups or distinguishing the user individually from other users.

Preferably, the adjusting is further based on at least one pre-set, wherein the at least one pre-set comprises at least one of: an equalizer; a genre; and a timbre. The pre-set may preferably be manually set by the user prior to the adjusting, or it may be a default pre-set set by an operator of the method.

Preferably, the sensory signal is a media signal, and the sensory signal is based on, preferably selected from, a plurality of media signals belonging to a predetermined media library of the user.

Preferably, the sensory signal comprises an audio signal, and the adjusting comprises at least one of:
amplifying or weakening audio amplitudes of the audio signal over at least 3, preferably at least 5, frequency bands: and
removing or adding at least one audio tone from or to at least one frequency band.

Preferably, the adjusting is performed in such a way that hearable audio distortion is prevented near edges of the frequency bands and that higher harmonic frequencies of the audio signal are maintained.

Preferably, the sensory signal comprises a video signal, and the adjusting comprises at least one of:
amplifying or weakening at least one of the following: a light output value; a colour value; a video framerate; a video blur; and a video focus;
removing or adding at least one video frame of the sensory signal; and
removing or adding at least one video component from or to at least one video frame of the sensory signal.

Preferably, the adjusting is based on different cut-off frequencies and/or different attenuation values for different individual users and/or for different groups of users.

Preferably, the sensory signal comprises a light signal, and the adjusting comprises at least one of:
changing a combination of electromagnetic frequencies of the light signal;
amplifying or weakening at least one light amplitude over at least one frequency range of the light signal; and
varying at least one light amplitude of the light signal over a time pattern.

Preferably, the method is performed repeatedly over a plurality of biofeedback sessions, each session having a duration of at least 5 minutes, preferably at least 10 minutes, more preferably at least 15 minutes, and wherein the plurality of biofeedback sessions spans at least a time period of 3 days, preferably at least 7 days, more preferably at least 28 days.

According to a third aspect according to the present disclosure, there is provided a method for providing a recommender system for recommending media content to a user, comprising: performing the method of any one of the above-described methods for biofeedback; and based on associations of the captured at least one bio-signal, the sensory signal, and the adjusted sensory signal, performing a machine learning process in order to train a recommender system configured for recommending media content to the user.

The skilled person will appreciate that analogous considerations and advantages may apply to the method as for the above-described system, mutatis mutandis.

According to a fourth aspect according to the present disclosure, there is provided a recommender system for recommending media content, characterized in that the recommender system has been trained according to the method for providing a recommender system.

The skilled person will appreciate that analogous considerations and advantages may apply to the recommender system as for the above-described system, mutatis mutandis.

According to a fifth aspect according to the present disclosure, there is provided a method for providing an equalizer, comprising: performing the method of any one of the above-described methods for biofeedback; and determining an equalizer based on the adjusting.

The skilled person will appreciate that analogous considerations and advantages may apply to the method as for the above-described system, mutatis mutandis.

According to a sixth aspect according to the present disclosure, there is provided a use of an equalizer provided by the above-described method for providing an equalizer.

The skilled person will appreciate that analogous considerations and advantages may apply to this use as for the above-described system, mutatis mutandis.

Advantageously, this allows to use an equalizer tailored to one user for another user, if the one user and the other user are sufficiently compatible, for example based on their belonging to the same user group.

According to a seventh aspect according to the present disclosure, there is provided a computer-readable data carrier, carrying a computer program comprising instructions that, when executed on at least one processor, cause the at least one processor to perform any one of the above-described methods.

The skilled person will appreciate that analogous considerations and advantages may apply to the computer-readable data carrier as for the above-described system, mutatis mutandis.

Brief description of the drawings & Detailed description

The embodiments described above will be more fully understood with the help of the appended drawings, in which like elements carry like reference signs, wherein:

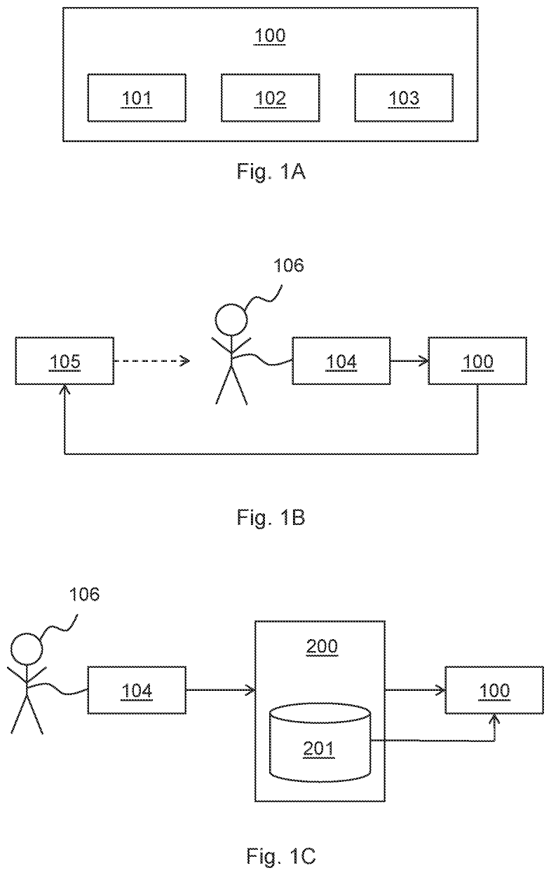

FIG. 1A schematically illustrates a system according to the present disclosure;

FIG. 1B schematically illustrates another system according to the present disclosure;

FIG. 1C schematically illustrates another system according to the present disclosure;

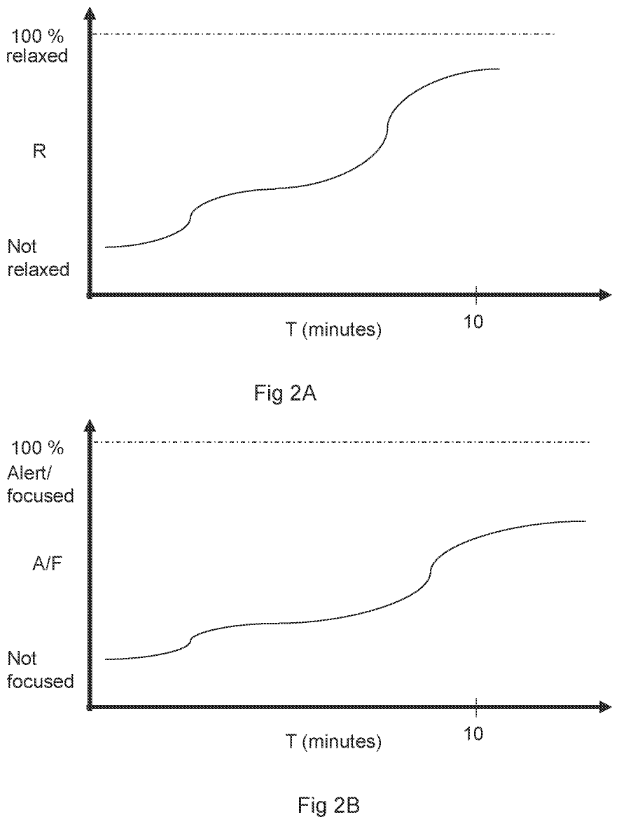

FIG. 2A schematically illustrates a graph of an example relation over time during a biofeedback session between characteristics of (real time or samples of) bio-signals (such as alpha and beta waves in brainwaves or the interbeat intervals in heart rate), either from one bio-signal or by combining the characteristics from a plurality of bio-signals and the actual value over time of a relaxation measure R;

FIG. 2B schematically illustrates a graph of another example relation over time during a biofeedback session between characteristics of (real time or samples of) bio-signals (such as alpha and beta waves in brainwaves or the interbeat intervals in heart rate), either from one bio-signal or by combining the characteristics from a plurality of bio-signals and the actual value over time of an Alertness/Focus measure A/F;

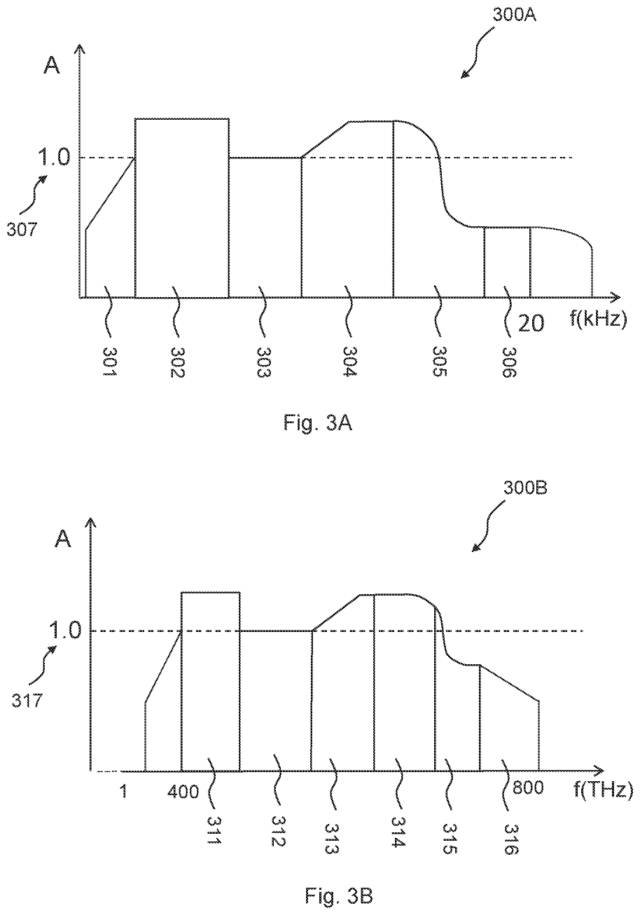

FIG. 3A schematically illustrates an example graph of a media equalizer, in particular for audio signals;

FIG. 3B schematically illustrates an example graph of a light equalizer;

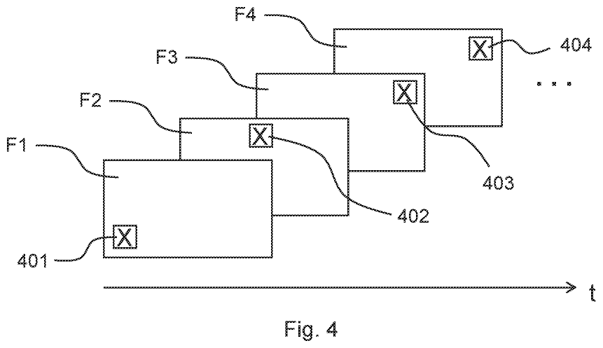

FIG. 4 schematically illustrates an example video signal comprising video frames over time;

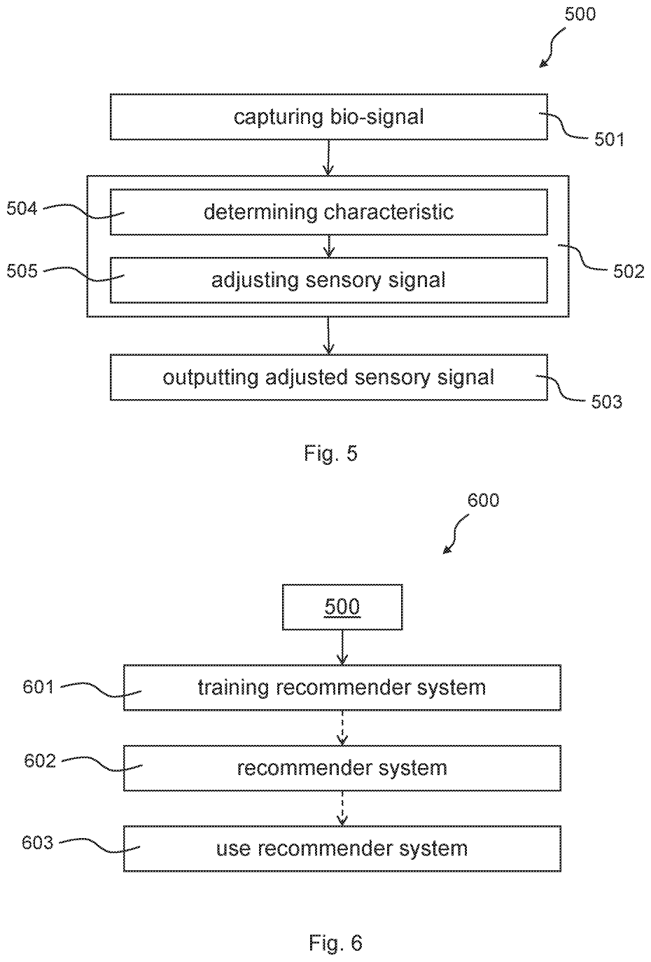

FIG. 5 schematically illustrates a method for biofeedback:

FIG. 6 schematically illustrates a method including a method of training a recommender system based on the method of FIG. 5 or on a further development of the method of FIG. 5, in order to produce a recommender system; and

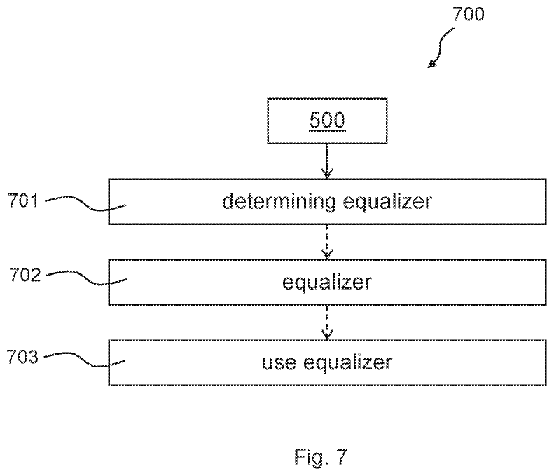

FIG. 7 schematically illustrates a method including a method of determining an equalizer based on the method of FIG. 5, in particular on an adjusting step of that method, or on a further development of the method of FIG. 5, in order to produce an equalizer.

FIG. 1A schematically illustrates a system 100 according to the present disclosure. System 100 comprises at least one input apparatus 101 configured for capturing at least one bio-signal of a user. The at least one bio-signal may be any of the bio-signals described above. Input apparatus 101 may itself be a sensor that is arranged for capturing the at least one bio-signal, or it may be an interface coupled to an external sensor (not shown) in order to receive and thus capture the at least one bio-signal from that external sensor. System 100 further comprises a processing module 102 configured for adjusting a sensory signal relative to a default setting of the sensory signal. Processing module 102 may e.g. be a logical software module incorporated on an electronic system comprising a processor and a memory. System 100 further comprises a signal interface 103 configured for outputting the adjusted sensory signal to a signal playback device (not shown). The signal interface may e.g. comprise a transmitter configured for transmitting the signal to the signal playback device, or it may be directly coupled to the signal playback device. The signal playback device may in a practical implementation be arranged for being perceivable to the user. The signal playback device may e.g. be an audio speaker or a video display or a combination thereof. Alternatively or additionally, the signal playback device may e.g. be a light generating system, dimmable light and/or a haptic device configured for tactile stimulation of the user and/or any other device capable of outputting a sensory output that the user can perceive with one or more of their senses. If the signal playback device is a haptic device such as a smartphone, then it is preferred to couple a vibration function of the haptic device to the bio-signal of the user, e.g. the breath rhythm of the user, in order to further improve the biofeedback.

The processing module 102 may be configured for said adjusting by: determining at least one characteristic based on the at least one bio-signal and subsequently adjusting the sensory signal based on the at least one characteristic, using a machine learning procedure or based on an output from a pre-trained machine learning system. The processing module 102 is further configured such that the determining spans a calibration period of at least 10, preferably at least 20, seconds prior to the adjusting.

The system 100 serves for biofeedback, in the sense that it takes a bio-signal from a user and outputs an adjusted sensory signal, which can be directed to that user.

FIG. 1B schematically illustrates another system 100 according to the present disclosure. System 100 of FIG. 1B may be a further development of system 100 of FIG. 1A, and therefore shares the same reference sign. FIG. 1B shows a user 106, to whom an optional sensor 104 is connected in some manner. Sensor 104 may e.g. be an EEG-sensor, or any type of sensor capable of sensing at least the desired type of bio-signal for system 100. One or more sensors may be used. Sensor 104 may be connected to system 100, e.g. to an input apparatus 101 of system 100, in order to provide the at least one bio-signal to system 100.

FIG. 1B further shows an optional signal playback device 105, arranged for being perceivable to the user 106. Said being perceivable is illustrated in this figure with a dashed line. Signal playback device 105 may e.g. be a signal playback device as described above with respect to FIG. 1A.

The system 100 serves for biofeedback analogously to the situation in FIG. 1A. FIG. 1B further shows how the feedback loop may be closed, via the signal playback device 105.

FIG. 1C schematically illustrates another system 100 according to the present disclosure. System 100 of FIG. 1B may be a further development of system 100 of FIG. 1A or of FIG. 1B, and therefore shares the same reference sign. FIG. 1B shows a user 106, to whom an optional sensor 104 is connected in some manner, analogously to the situation in FIG. 1B.

FIG. 1C further shows an optional intermediary device 200, e.g. a smartphone or a personal computer. The intermediary device 200 may relay the at least one bio-signal to system 100, e.g. to at least one input apparatus 101 of system 100.

FIG. 1C further shows media library 201, which may optionally be stored on intermediary device 200, and which may hold preferred media content of the user 106. Media library 201 may e.g. be a personal music or video or multimedia library on a smartphone or on a network attached storage in a local area network of the user 106. Alternatively, media library 201 may be stored elsewhere, e.g. in the cloud, as a streaming media library of the user 106. Media library 201 may be connected to system 100, in particular to processing module 102 of system 100, in order to provide a default setting of the sensory signal. It will be appreciated that this advantageously allows system 100 to operate on preferred media content of the user 106, thus improving feelings of comfort for the user 106.

FIG. 2A schematically illustrates a graph of an example relation over time during a biofeedback session between characteristics of (real time or samples of) bio-signals (such as alpha and beta waves in brainwaves or the interbeat intervals in heart rate), either from one bio-signal or by combining the characteristics from a plurality of bio-signals and the actual value over time of a relaxation measure R.

This relation is based on a mathematical computation of the input from the characteristics of (real-time or samples of) bio-signals resulting into this actual R value. This actual R value together with pre-set values (coming from the user's/target group preferences and/or from machine learning) determines a cumulative reward function applied on the original sensory signal (e.g. music, light, . . . ) during a biofeedback session with possible adjusting examples as given in FIG. 3A (adjusting audio sensory signal) and FIG. 3B (adjusting light sensory signal).

FIG. 2B schematically illustrates a graph of another example relation over time during a biofeedback session between characteristics of (real time or samples of) bio-signals (such as alpha and beta waves in brainwaves or the interbeat intervals in heart rate), either from one bio-signal or by combining the characteristics from a plurality of bio-signals and the actual value over time of an Alertness/Focus measure A/F.

This relation is based on a mathematical computation of the input from the characteristics of (real-time or samples of) bio-signals resulting into this actual A/F value. This actual A/F value together with pre-set values (coming from the user's/target group preferences and/or from machine learning) determines a cumulative reward function applied on the original sensory signal (e.g. music, light, . . . ) during a biofeedback session with possible adjusting examples as given in FIG. 3A (adjusting audio sensory signal) and FIG. 3B (adjusting light sensory signal).

FIG. 3A schematically illustrates an example graph of a media equalizer 300A. In this example, the media equalizer 300A takes the form of a function of frequency f (expressed in kilohertz) to amplitude scaling factor A, wherein the amplitude scaling factor A is expressed in relation to a default setting, e.g. 1.0 (307). It will be appreciated that the media equalizer may be applied to an audio signal, e.g. by multiplying an audio amplitude of the audio signal with the amplitude scaling factor for a given frequency band. The figure shows that there may be multiple frequency bands 301-306 in the hearable audio frequency band (20 Hz-20 kHz), in this example six although there could be fewer or more, wherein an amplitude scaling factor is defined.

Among all kinds of media, especially music has been scientifically proven to be a direct means to influence the human mind via the hearing senses and parasympathetic nervous system. This contributes to self-awareness, self-regulation. The long term effect on stress relief of these approaches depends on how well users adhere to them and how much effort that costs. Other types of media signals detectable by human senses like eyesight or touch may have different or similar impact.

For frequency band 301, a linear increasing amplitude scaling factor of less than 1.0 is chosen; i.e. the audio amplitude of the audio signal will be linearly weakened over frequency band 301.

In contrast, for frequency band 302, an amplitude scaling factor of more than 1.0 is chosen, i.e. the audio amplitude of the audio signal will be amplified over all of frequency band 302.

There may optionally also be one or more frequency bands over which the amplitude scaling factor is maintained at 1.0, in this example frequency band 303. This means that over this frequency band, the audio amplitude of the audio signal will not be adjusted via audio amplitude scaling.

There may optionally also be more complex definitions of the amplitude scaling factor. E.g. for frequency band 304 a linear piecewise function is defined, and for frequency band 305 a continuous curve is defined.

For frequency band 306, an amplitude scaling factor of 0.5 is chosen, i.e. the audio amplitude of the audio signal will be weakened over all of frequency band 306

Optionally, one or more audio tones may be removed or added from or to at least one frequency band (not shown).

Optionally, any one or more of beat, fading and stereo levels may also be adjusted.

Optionally, one or more audio tones (also higher harmonics) may be removed or added from or to at least one frequency band outside the hearable audio frequency band (beyond frequency band 306 or below frequency band 301).

FIG. 3B schematically illustrates an example graph of a light equalizer 300B for adjusting a light signal of an optional light generating system (not shown). In this example, the light equalizer 300B takes the form of a function of frequency f (expressed in terahertz) to amplitude scaling factor A. The amplitude scaling factor A is expressed in relation to a default setting, e.g. 1.0 (317). The figure shows that there may be multiple electromagnetic frequency bands 311-316 in the visible spectrum, being the portion of the larger electromagnetic spectrum that humans can see from around 400 THz (red) to around 800 THz (violet). In this example there are six such bands in the visible spectrum and one below the visible spectrum, although there could be fewer or more, wherein an amplitude scaling factor is defined.

The visible electromagnetic frequency bands (or optionally invisible electromagnetic frequency bands which are detectable, and may possibly be harmful) can be adjusted by changing the combination of electromagnetic frequencies i.e. polychromatic light (associated with perception of colour) and amplitude of light (associated with human experience of brightness or intensity of colour) according to the process as described in the previous FIG. 3A, method 100 and system 100 and illustrated in FIG. 2A and FIG. 2B. In other words, the adjusting of the light signal may comprise at least one of:
changing a combination of electromagnetic frequencies of the light signal;
amplifying or weakening at least one light amplitude over at least one frequency range of the light signal; and
varying at least one light amplitude of the light signal over a time pattern, e.g. in order to obtain a flickering pattern of the light signal.

In another example embodiment, the sensory signal may be a light signal, e.g. a dynamic or ambient lighting, whose amplitude may be increased or decreased, e.g. according to the light equalizer 300B described in FIG. 3B, and/or whose colour value may be adapted, and/or which may have other properties that can be adjusted, according to the present disclosure.

Also, a plurality of types of sensory signals may be combined, e.g. an audio signal and a light signal, or a tactile signal and an audio signal, or a video signal and a smell signal, etc.

FIG. 4 schematically illustrates an example video signal comprising video frames F1-F4, over time t. Video frames are pictures that together constitute a video.

In the video frames F1-F4, an object 401-404 is shown, represented here abstractly as a cross in a rectangle, which may be of interest to the user. For example, the object 401-404 may be an image region of a person or a pet or a likable inanimate object. The image region of the objects 401-404 in the respective video frames F1-F4 may be adjusted relative to its default setting, for example by amplifying its light output value, by amplifying the colour value, e.g. to improve a dynamic contrast of colour values of the object 401-404, and/or by amplifying a video focus of the image region of the object 401-404. Additionally or alternatively, other regions of the video frames F1-F4 than the image region of the object 401-404 may be adjusted relative to their default settings, for example, a video focus of regions other than the object 401-404 may be weakened, a colour value may be weakened, and/or a light output value may be weakened. It may be an aim in this context to strengthen the user's perception of the person, pet or likable inanimate object in order to further improve the biofeedback.

In other words, any one or more of the following operations may optionally be performed on the video signal: amplifying or weakening at least one of the following: a light output value; a colour value; a video framerate; a video blur; and a video focus; and removing or adding at least one video component from or to at least one video frame of the sensory signal. In this context, a video component can be any technical part of the video signal that is usable for technically interpreting the video signal.

Optionally, one or more video frames may be removed from the video signal, e.g. video frame F3 may be removed. Optionally, one or more video frames may be added to the video signal, e.g. a new video frame may be added between video frame F3 and video frame F4. In that case, it is preferred to either take one of the neighboring video frames F3 and F4 to be the new video frame, or to produce a new video frame that is a transition video frame between video frames F3 and F4, e.g. as an interpolation of those two video frames.

FIG. 5 schematically illustrates a method 500 for biofeedback. The method comprises: capturing 501 at least one bio-signal of a user; adjusting 502 a sensory signal relative to a default setting of the sensory signal; and outputting 503 the adjusted sensory signal to a signal playback device arranged for being perceivable to the user.

The step of adjusting 502 the sensory signal comprises: determining 504 at least one characteristic based on the at least one bio-signal; and adjusting 505 the sensory signal based on the at least one characteristic, using a machine learning procedure or based on an output from a pre-trained machine learning system. The determining spans a calibration period of at least 10, preferably at least 20, seconds prior to the adjusting.

An example procedure of determining at least one characteristic based on the at least one bio-signal and of adjusting the sensory signal based on the at least one characteristic, using a machine learning procedure or based on an output from a pre-trained machine learning system, is described in the following. The example below refers specifically to an audio signal as the sensory signal, but the considerations may also be applicable to different types of signals and also to combinations of multiple types of signals.

Prior to or at the beginning of a biofeedback session, a default equalizer may be selected, for example based on a generic default, or preferably on a user-specific or user group-specific default, which defaults have been determined prior to the biofeedback session, e.g. pre-trained by a machine learning system, in order to generate a default equalizer ahead of time. During the biofeedback session, i.e. during a time of operation of the system for biofeedback, a machine learning agent may control the equalizer, i.e. may adjust the equalizer, in order to satisfy a machine learning goal, e.g. a goal to optimize a cumulative reward function. In this sense, a machine learning procedure can be used in order to learn a relation between one or more characteristics and one or more adjustments, wherein the equalizer is continuously adjusted and wherein the impact of such adjustments is determined via the biofeedback loop. This allows the method to base the adjusting on the at least one characteristic using a machine learning procedure.

Alternatively, adjusting the sensory signal may be based on the at least one characteristic, based on an output from a pre-trained machine learning system, if no machine learning agent is dynamically controlling and learning from the adjusting, by providing a pre-trained machine learning system and using its output as a static default equalizer. The pre-trained machine learning system may itself be trained beforehand using all steps of a method according to the present disclosure except based not on another machine learning system, but e.g. on a manually pre-defined default equalizer.

Advantageously, the cumulative reward function may represent a measure for user relaxation or user alertness/focus, e.g. by applying a mathematical computation on the characteristics of one real-time measured bio-signal (such as EEG brainwaves or HRV) or by combining a plurality of bio-signals of the user, such as brainwaves and heart rate variability.

The agent may be provided with the at least one bio-signal in the advantageous form of the cumulative reward function, preferably on an ongoing basis during the biofeedback session. The agent may be configured to interpret the cumulative reward function as a characteristic of the user, e.g. whether the user is relaxed or tensed.

Alternatively or additionally, the agent may be configured to derive at least one signal characteristic of the at least one bio-signal and may be configured to base the adjustments on the at least one derived signal characteristic.

By making adjustments to the equalizer, and thus ensuring that the audio signal is in turn adjusted (because the equalizer will be applied to the audio signal before perception by the user) and by determining impact of those adjustments on the cumulative reward function, the machine learning agent may be rewarded or punished in order to learn which adjustments lead to the greatest expected reward, i.e. reinforcement learning.]

Note that the above description of an example procedure using machine learning is merely one option. The skilled person will appreciate that other types of machine learning approaches may also be used.

In a further developed example, the default equalizer to be used in a biofeedback session may be determined prior to the biofeedback session, for example by clustering users into k various user groups, using a clustering algorithm such as k-means clustering. For each user group, an optimal equalizer may be determined, for example using the above described procedure starting from a generic default equalizer. Then, in the present biofeedback session, a user may be assigned to a user group among the k various user groups and the respective equalizer of that user group may be selected as a user group-specific default.

FIG. 6 schematically illustrates a method 600 including a method 601 of training a recommender system based on method 500 of FIG. 5 or on a further development of method 500, in order to produce a recommender system 602. The recommender system 602 is a product of the method 601 and is therefore shown with a dashed line. The recommender system 602 may subsequently be used in a method 603 of using the recommender system, which relationship is also shown with a dashed line.

Based on associations of the captured at least one bio-signal, the sensory signal, and the adjusted sensory signal, a machine learning process may be performed in order to train the recommender system configured for recommending media content to the user. A particular example of recommended media content may e.g. be a sequence of audio signals, e.g. music tracks.

In a further developed example, the machine learning process may comprise training an ensemble/hybrid recommender, i.e. a recommender system configured to combine outputs of a plurality of recommender systems.

In a particular example, the recommender systems may use collaborative filtering, e.g. item-to-item collaborative filtering and/or content-based filtering, based on e.g. the above-described measure for user relaxation, a particular sequence of audio signals chosen to be fed back to the user, and the adjustments that have been made to said audio signals.

It is preferred to keep training the recommender system in the above-described manner. Additionally, it may be preferred to poll the user for a score on the recommended media content, and to further base the method of training the recommender system on the polled score.

In a practical example, in order to mitigate sparsity of matrices used in the recommender system and thus to mitigate reduced accuracy of recommended media content, it may be preferred to reduce an amount of initial candidate recommendations using item filters, e.g. based on popular music, on user or user group preferences, and/or on expert opinion.

FIG. 7 schematically illustrates a method 700 including a method 701 of determining an equalizer based on method 500, in particular on an adjusting step of that method, of FIG. 5 or on a further development of method 500, in order to produce an equalizer 702, e.g. the equalizer 300A of FIG. 3A (or similar for the light equalizer 300B of FIG. 3B). The equalizer 702 is a product of the method 701 and is therefore shown with a dashed line. The equalizer 702 may subsequently be used in a method 703 of using the equalizer, which relationship is also shown with a dashed line.

The equalizer 702 may for example be produced according to the above described procedure using a machine learning agent, although the skilled person will appreciate that other approaches may also be used.

WO2012080962A1 discloses a system for providing biofeedback to a person, comprising a source for generating a source signal, a transducer for generating a measurement signal in response to a physiological parameter indicative for mental relaxation of the person, a filter for variably filtering the source signal via modifying a cut-off frequency in response to the measurement signal, and an interface for providing a biofeedback signal to the person on the basis of the source signal as variably filtered by the filter.

The above-cited disclosure requires a filter for adapting a cut-off frequency to a measurement signal indicative for mental relaxation and subsequently filtering a source signal by such adaptive filter and by basing the biofeedback signal on such variably filtered source signal.

Also, in the above-cited disclosure, there is no determination of at least one characteristic based on the at least one bio-signal, thus limiting the basis for the filtering. Moreover, the biofeedback signal is not based on media signals belonging to a predetermined media library of the user, and therefore cannot achieve optimal effect.

In the context of the present disclosure, the term “focus” may be understood to encompass both mental relaxation and mental performance.

In embodiments according to the present disclosure, it may be considered to provide the unadjusted media signal, i.e. the media signal in its default setting, to the user, after providing the adjusted media signal, either within the duration of a biofeedback session or after the biofeedback session, in order to induce a respondent conditioning. In this manner, the user may have even easier access to the benefits of the present disclosure.

Advantageously, in some embodiments according to the present disclosure, instructions may be provided to the user regarding meditation procedures and/or exercise procedures. This has the benefit of further improving the wellbeing of the user.

In specific example embodiments of the system and method according to the present disclosure, the user may belong to a user group of pregnant women. Pregnancy is associated with profound cardiovascular adaptation with altered cardiac autonomic balance. It can be studied by heart rate variability (HRV) which indicates beat to beat RR interval variation on ECG. Notably the second trimester is associated with major decline in HRV. There is a global HRV reduction in normal pregnancy across all trimesters, associated with primiparity. This indicates pregnancy as a significant risk with reference to altered cardiac balance and use of HRV as a good tool to assess the same.

It is therefore desirable to tailor the approach to the user group of pregnant women.

Advantageously, this may be done by adjusting sensory signals according to the average reduction a woman will experience during pregnancy, especially during the first pregnancy and during the second trimester.

In embodiments according to the present disclosure, machine learning may be applied based on labelled data of previously investigated pregnant women, with the goal of returning the HRV measurement to pre-pregnancy values or as much as is reasonably expected during pregnancy with a reference to the least affected pregnant individuals (reference group).

Furthermore, pregnancy is sometimes related to hearing loss and vestibular complaints; studies have found that over half of pregnant women suffer from tinnitus. Advantageously, embodiments according to the present disclosure may therefore take into account differences in biofeedback between pregnant women with and without symptoms of tinnitus.

Advantageously, by increasing HRV, more physical resilience may be achieved and mental wellbeing may be improved, possibly with direct effect on the unborn child, hence positively influencing the deleterious effects of pregnancy on the cardiovascular system.

Of course, the above advantages for pregnant women with tinnitus may also apply to other types of users with tinnitus, and as well to other types of users with other “hearing loss” symptoms. For example, in other example embodiments of the system and method according to the present disclosure, high-frequency hearing loss amongst the elderly may be taken into account, wherein a region, typically a broad high-frequency region, within the hearable spectrum of sound is affected.

Advantageously, the biofeedback according to the present disclosure may involve adjustments to the sensory signal taking into account this phenomenon, for example by optimizing the adjustments to focus on the unaffected regions of the hearable sound spectrum.

1. A system for biofeedback, comprising:
at least one input apparatus configured for capturing at least one bio-signal of a user;
a processing module configured for adjusting a sensory signal relative to a default setting of the sensory signal;
a signal interface configured for outputting the adjusted sensory signal to a signal playback device arranged for being perceivable to the user;
wherein the adjusting comprises:
determining at least one characteristic based on the at least one bio-signal;
adjusting the sensory signal based on the at least one characteristic, using a machine learning procedure or based on an output from a pre-trained machine learning system; and
wherein the determining spans a calibration period of at least 10, preferably at least 20, seconds prior to the adjusting; and
characterized in that the processing module is configured for:
defining the at least one characteristic determined at the end of the calibration period as a reliable level of the at least one characteristic;
during the adjusting, detecting whether a reliability of the at least one characteristic becomes less than the reliable level; and
upon detecting that the reliability of the at least one characteristic becomes less than the reliable level, temporarily suspending the adjusting of the sensory signal until another calibration period of at least 10, preferably at least 20, seconds has passed.

2. (canceled)

3. The system of claim 1, wherein the at least one bio-signal comprises at least one of the following:
brainwaves;
a muscular activity;
a heart rate;
a heart rate variability;
a breath rhythm;
a blood pressure;
a body temperature;
a gas content of blood;
a perspiration;
an electrodermal activity;
a facial expression;
a dexterity performance parameter; and
a voice pattern.

4. The system of claim 1, wherein the at least one characteristic is a signal characteristic of the at least one bio-signal and/or wherein the at least one characteristic is a user characteristic selected for distinguishing a group to which the user belongs among a plurality of groups or distinguishing the user individually from other users.

5. The system of claim 1, wherein the adjusting is further based on at least one pre-set, wherein the at least one pre-set comprises at least one of: an equalizer; a genre; and a timbre.

6. The system of claim 1, wherein the sensory signal is a media signal, and wherein the sensory signal is based on, preferably selected from, a plurality of media signals belonging to a predetermined media library of the user.

7. The system of claim 1, wherein the sensory signal comprises an audio signal, and wherein the adjusting comprises at least one of:
amplifying or weakening audio amplitudes of the audio signal over at least 3, preferably at least 5, frequency bands; and
removing or adding at least one audio tone from or to at least one frequency band.

8. The system of claim 7, wherein the adjusting is performed in such a way that hearable audio distortion is prevented near edges of the frequency bands and that higher harmonic frequencies of the audio signal are maintained.

9. The system of claim 1, wherein the sensory signal comprises a video signal, and wherein the adjusting comprises at least one of:
amplifying or weakening at least one of the following: a light output value; a colour value; a video framerate; a video blur; and a video focus;
removing or adding at least one video frame of the sensory signal; and
removing or adding at least one video component from or to at least one video frame of the sensory signal.

10. The system of claim 7, wherein
the adjusting is based on different cut-off frequencies and/or different attenuation values for different individual users and/or for different groups of users.

11. The system of any previous claim 1, wherein the sensory signal comprises a light signal, and wherein the adjusting comprises at least one of:
changing a combination of electromagnetic frequencies of the light signal;
amplifying or weakening at least one light amplitude over at least one frequency range of the light signal; and
varying at least one light amplitude of the light signal over a time pattern.

12. A method for biofeedback, comprising:
capturing at least one bio-signal of a user;
adjusting a sensory signal relative to a default setting of the sensory signal; and
outputting the adjusted sensory signal to a signal playback device arranged for being perceivable to the user;
wherein the adjusting comprises:
determining at least one characteristic based on the at least one bio-signal; and
adjusting the sensory signal based on the at least one characteristic, using a machine learning procedure or based on an output from a pre-trained machine learning system; and
that wherein the determining spans a calibration period of at least 10, preferably at least 20, seconds prior to the adjusting; and
characterized by comprising:
defining the at least one characteristic determined at the end of the calibration period as a reliable level of the at least one characteristic;
during the adjusting, detecting whether a reliability of the at least one characteristic becomes less than the reliable level; and
upon detecting that the reliability of the at least one characteristic becomes less than the reliable level, temporarily suspending the adjusting of the sensory signal until another calibration period of at least 10, preferably at least 20, seconds has passed.

13. (canceled)

14. The method of claim 12, wherein the at least one bio-signal comprises at least one of the following:
brainwaves;
a muscular activity;
a heart rate;
a heart rate variability;
a breath rhythm;
a blood pressure;
a body temperature;
a gas content of blood;
a perspiration;
an electrodermal activity;
a facial expression;
a dexterity performance parameter; and
a voice pattern.

15. The method of claim 12, wherein the at least one characteristic is a signal characteristic of the at least one bio-signal and/or wherein the at least one characteristic is a user characteristic selected for distinguishing a group to which the user belongs among a plurality of groups or distinguishing the user individually from other users.

16. The method of claim 12, wherein the adjusting is further based on at least one pre-set, wherein the at least one pre-set comprises at least one of: an equalizer; a genre; and a timbre.

17. The method of claim 12, wherein the sensory signal is a media signal, and wherein the sensory signal is based on, preferably selected from, a plurality of media signals belonging to a predetermined media library of the user.

18. The method of claim 12, wherein the sensory signal comprises an audio signal, and wherein the adjusting comprises at least one of:
amplifying or weakening audio amplitudes of the audio signal over at least 3, preferably at least 5, frequency bands; and
removing or adding at least one audio tone from or to at least one frequency band.

19. The method of claim 18, wherein the adjusting is performed in such a way that hearable audio distortion is prevented near edges of the frequency bands and that higher harmonic frequencies of the audio signal are maintained.

20. The method of claim 12, wherein the sensory signal comprises a video signal, and wherein the adjusting comprises at least one of:
amplifying or weakening at least one of the following: a light output value; a colour value; a video framerate; a video blur; and a video focus;
removing or adding at least one video frame of the sensory signal; and
removing or adding at least one video component from or to at least one video frame of the sensory signal.

21. The method of claim 18, wherein the adjusting is based on different cut-off frequencies and/or different attenuation values for different individual users and/or for different groups of users.

22. The method of claim 12, wherein the sensory signal comprises a light signal, and wherein the adjusting comprises at least one of:
changing a combination of electromagnetic frequencies of the light signal;
amplifying or weakening at least one light amplitude over at least one frequency range of the light signal; and
varying at least one light amplitude of the light signal over a time pattern.

23. The method of claim 12, performed repeatedly over a plurality of biofeedback sessions, each session having a duration of at least 5 minutes, preferably at least 10 minutes, more preferably at least 15 minutes, and wherein the plurality of biofeedback sessions spans at least a time period of 3 days, preferably at least 7 days, more preferably at least 28 days.

24. A method for providing a recommender system for recommending media content to a user, comprising:
performing the method of claim 12; and
based on associations of the captured at least one bio-signal, the sensory signal, and the adjusted sensory signal, performing a machine learning process in order to train a recommender system configured for recommending media content to the user.

25. A recommender system for recommending media content, characterized in that the recommender system has been trained according to the method of claim 24.

26. A method for providing an equalizer, comprising:
performing the method of claim 12; and
determining an equalizer based on the adjusting.

27. Use of an equalizer provided by the method of claim 26.

28. A computer-readable data carrier, carrying a computer program comprising instructions that, when executed on at least one processor, cause the at least one processor to perform the method of claim 12.
